(19) United States
(12) Patent Application Publication
(10) Pub. No.: US 2013/0012813 A1
(43) Pub. Date: Jan. 10, 2013

(54) IMAGE PROCESSING APPARATUS AND X-RAY DIAGNOSTIC APPARATUS

(76) Inventor: Takuya SAKAGUCHI, Utsunomiya-shi (JP)

(21) Appl. No.: 13/611,427

(22) Filed: Sep. 12, 2012

Related U.S. Application Data
(63) Continuation of application No. PCT/JP12/65891, filed on Jun. 21, 2012.

(30) Foreign Application Priority Data
Jun. 23, 2011 (JP) 2011-139562

(51) Int. Cl. G06K 9/00; A61B 6/00

(52) U.S. Cl. 600/431; 382/132

(57) ABSTRACT

According to one embodiment, an image processing apparatus includes a storage unit, an image generation unit, and a display control unit. The storage unit stores the first X-ray fluoroscopy image with a cardiac tissue of an object being contrast-enhanced by a contrast medium and the second X-ray fluoroscopy image with a cardiac lumen of the object being contrast-enhanced by the contrast medium. The image generation unit generates an image by combining the first X-ray fluoroscopy image and the second X-ray fluoroscopy image which are stored in the storage unit. The display control unit causes a display unit to display the image generated by the image generation unit.

### CROSS REFERENCE TO RELATED APPLICATIONS

This application is a Continuation Application of PCT Application No. PCT/JP2012/065891, filed Jun. 21, 2012 and based upon and claiming the benefit of priority from Japanese Patent Application No. 2011-139562, filed Jun. 23, 2011, the entire contents of all of which are incorporated herein by reference.

### FIELD

Embodiments described herein relate generally to an image processing apparatus and an X-ray diagnostic apparatus.

### BACKGROUND

With recent advances in regenerative medical techniques, there is being established a therapy (cell therapy) for recovering the myocardial motion by promoting cell growth and cell activation by directly administering stem cells or cell growth factors to an ischemia/infarction region of a cardiac muscle.

As techniques of administering stem cells or the like to an ischemia/infarction region in this type of therapy, there have been proposed a surgical technique, a technique of injecting stem cells or the like into a coronary artery through a catheter, a technique of injecting stem cells or the like from the ventricular lumen side through a catheter, and the like.

Either of these techniques needs to clarify an ischemia/infarction region in advance and position the distal end of a catheter by moving the catheter so as to inject stem cells or the like.

The above ischemia/infarction region is comprehended based on, for example, the myocardial perfusion images captured by an X-ray diagnostic apparatus. A myocardial perfusion image is an X-ray fluoroscopy image captured upon inserting a catheter into a coronary artery of the heart and injecting a contrast medium through the catheter. This myocardial perfusion image depicts the myocardial tissue contrast-enhanced by the contrast medium, and hence allows to comprehend a myocardial region with normal perfusion.

In a myocardial perfusion image, a region which a contrast medium can reach, i.e., a region to which oxygen is supplied by blood, is visualized, whereas no contrast medium flows into an ischemia/infarction region. This makes it impossible to accurately comprehend the distribution of the regions. That is, it is not possible to directly discriminate whether the region which is not contrast-enhanced in the myocardial perfusion image is not the myocardial tissue or the myocardial tissue which is not perfused. Under the circumstances, therefore, it is difficult to accurately comprehend an ischemia/infarction region based on an X-ray fluoroscopy image or to accurately administer stem cells or the like to the ischemia/infarction region.

### BRIEF DESCRIPTION OF THE DRAWINGS

FIG. 1 is a block diagram showing the arrangement of an X-ray diagnostic apparatus according to the first embodiment.

FIG. 2 is a functional block diagram of an image processing unit in the same embodiment.

FIG. 3 is a schematic view showing a procedure for image processing in the same embodiment.

FIG. 4 is a flowchart for explaining operation in the same embodiment.

FIG. 5 is a view for explaining a technique of extracting an X-ray fluoroscopy image group in the same embodiment.

FIG. 6 is a functional block diagram of an image processing unit in the second embodiment.

FIG. 7 is a schematic view showing a procedure for image processing in the second embodiment.

FIG. 8 is a schematic view showing a procedure for image processing in the second embodiment.

FIG. 9 is a schematic view showing a procedure for image processing in the second embodiment.

FIG. 10 is a view for explaining a procedure for specifying a contrast-enhanced region in the second embodiment.

FIG. 11 is a flowchart for explaining operation in the second embodiment.

FIG. 12 is a flowchart for explaining operation in the second embodiment.

FIG. 13 is a functional block diagram of an image processing unit in the third embodiment.

FIG. 14 is a schematic view showing a procedure for image processing in the third embodiment.

FIG. 15 is a view for explaining a technique of specifying an ischemia region in the third embodiment.

FIG. 16 is a flowchart for explaining operation in the third embodiment.

### DETAILED DESCRIPTION

In general, according to one embodiment, an image processing apparatus includes a storage unit, an image generation unit, and a display control unit. The storage unit stores the first X-ray fluoroscopy image with a cardiac tissue of an object being contrast-enhanced by a contrast medium and the second X-ray fluoroscopy image with a cardiac lumen of the object being contrast-enhanced by the contrast medium. The image generation unit generates an image by combining the first X-ray fluoroscopy image and the second X-ray fluoroscopy image which are stored in the storage unit. The display control unit causes a display unit to display the image generated by the image generation unit.

Several embodiments will be described below with reference to the accompanying drawings.

Note that each embodiment will exemplify a case in which an X-ray diagnostic apparatus incorporates an image processing apparatus.

### First Embodiment

The first embodiment will be described first.

### [Overall Arrangement of X-ray Diagnostic Apparatus]

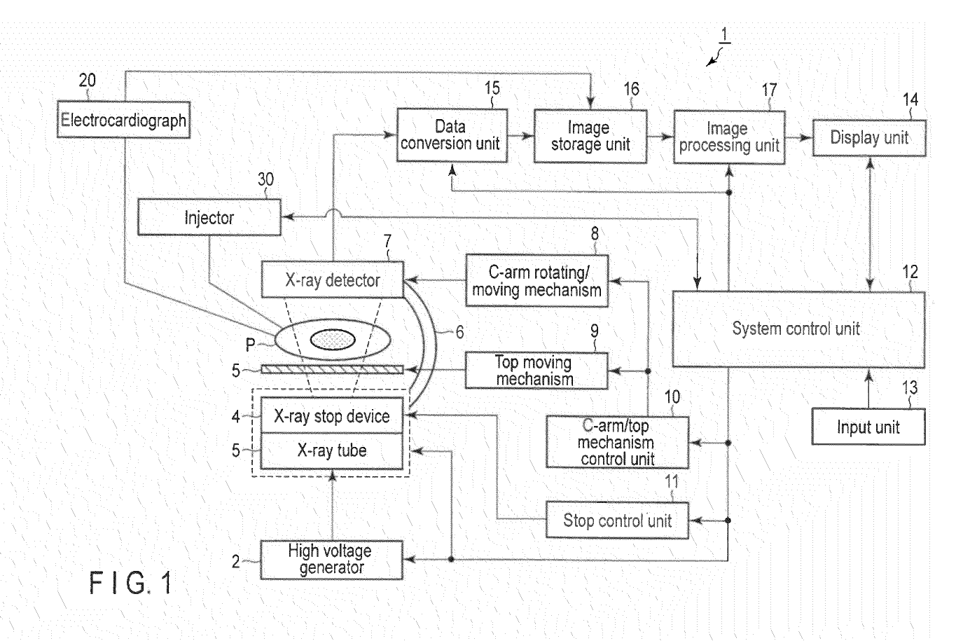

FIG. 1 is a block diagram showing the arrangement of an X-ray diagnostic apparatus 1 according to this embodiment.

As shown in FIG. 1, the X-ray diagnostic apparatus 1 according to this embodiment includes a high voltage generator 2, an X-ray tube 3, an X-ray stop device 4, a top 5, a C-arm 6, an X-ray detector 7, a C-arm rotating/moving mechanism 8, a top moving mechanism 9, a C-arm/top mechanism control unit 10, a stop control unit 11, a system control unit 12, an input unit 13, a display unit 14, a data conversion unit 15, an image storage unit 16, and an image processing unit 17.

An electrocardiograph 20 and an injector 30 are connected to the X-ray diagnostic apparatus 1 according to this embodiment.

The electrocardiograph 20 acquires the electrocardiographic waveform of an object P, and outputs the acquired electrocardiographic waveform together with time information to the image storage unit 16 and the like.

The injector 30 is a device for injecting a contrast medium through a catheter inserted into the object P. The injector 30 may inject a contrast medium in accordance with an instruction from the system control unit 12 or an instruction input by direct operation by the operator with the injector 30.

The high voltage generator 2 generates a high voltage to be applied to the X-ray tube 3. The X-ray tube 3 generates X-rays based on the high voltage applied from the high voltage generator 2.

The X-ray stop device 4 is a device for focusing the X-rays generated from the X-ray tube 3 so as to selectively irradiate a region of interest of the object P with the X-rays. For example, the X-ray stop device 4 includes four slidable aperture blades, and focuses X-rays by sliding these aperture blades.

The top 5 is a bed on which the object P is placed, and is placed on a bed (not shown).

The X-ray detector 7 includes a plurality of X-ray detection elements which detect the X-rays transmitted through the object P. These X-ray detection elements respectively convert the X-rays transmitted through the object P into electrical signals and store them.

The C-arm 6 holds the X-ray tube 3, the X-ray stop device 4, and the X-ray detector 7 so as to make them face each other through the object P.

The C-arm rotating/moving mechanism 8 is a device for rotating and moving the C-arm 6. The top moving mechanism 9 is a device for moving the top 5. The C-arm/top mechanism control unit 10 controls the C-arm rotating/moving mechanism 8 and the top moving mechanism 9 to adjust the rotation amount and movement amount of the C-arm 6 and the movement amount of the top 5.

The stop control unit 11 controls the irradiation range of X-rays by adjusting the opening of the aperture blades of the X-ray stop device 4.

The data conversion unit 15 reads out the charge stored in the X-ray detector 7 in synchronism with the application of an X-ray pulse, generates an X-ray fluoroscopy image by converting the readout electrical signal into digital data, and outputs the generated X-ray fluoroscopy image to the image storage unit 16.

The image storage unit 16 stores the X-ray fluoroscopy image output from the data conversion unit 15 in association with the imaging time. The image storage unit 16 also stores phase information with the electrocardiographic waveform output from the electrocardiograph 20 being associated with time information. Referring to the imaging time associated with this phase information and the X-ray fluoroscopy image can specify a cardiac phase corresponding to each X-ray fluoroscopy image stored in the image storage unit 16. The image storage unit 16 also stores the contrast medium injection start time of the injector 30. The apparatus notifies the image storage unit 16 via the system control unit 12 of this contrast medium injection start time when, for example, the injector 30 starts injecting a contrast medium.

The image processing unit 17 performs various kinds of image processing for each X-ray fluoroscopy image stored in the image storage unit 16. The function of the image processing unit 17 will be described in detail later.

The input unit 13 includes a mouse, keyboard, buttons, trackball, and joystick which are used to input various kinds of commands and information by an operator such as a doctor or technician who operates the X-ray diagnostic apparatus 1, and outputs commands and information input with these devices to the system control unit 12.

The display unit 14 includes a monitor such as an LCD (Liquid Crystal Display) or CRT (Cathode Ray Tube), and displays a GUI (Graphical User Interface) for accepting inputs from the operator via the input unit 13, an X-ray fluoroscopy image stored in the image storage unit 16, the X-ray fluoroscopy image processed by the image processing unit 17, and the like.

The system control unit 12 controls the overall operation of the X-ray diagnostic apparatus 1. That is, the system control unit 12 controls the high voltage generator 2, the C-arm/top mechanism control unit 10, the stop control unit 11, and the like, based on commands and the like from the operator which are input via the input unit 13, to perform adjutment of the dose of X-rays with which the object P is irradiated, ON/OFF control of X-ray irradiation, adjustment of the rotation/movement of the C-arm 6, movement adjustment of the top 5, and the like.

The system control unit 12 controls the data conversion unit 15 and the image processing unit 17 based on commands and the like from the operator which are input via the input unit 13. The system control unit 12 performs control for making the display unit 14 display the above GUI, an X-ray fluoroscopy image stored in the image storage unit 16, the X-ray fluoroscopy image processed by the image processing unit 17, and the like.

Using the X-ray diagnostic apparatus 1 with the above arrangement can obtain an X-ray fluoroscopy image, for example a myocardial perfusion image of the object P and a cardiac lumen contrast-enhanced image (a left ventricular contrast-enhanced image in this embodiment).

More specifically, the apparatus obtains a myocardial perfusion image by making the injector 30 inject a contrast medium from a catheter and continuously capturing X-ray fluoroscopy images with a region of interest including the heart while inserting the catheter into a coronary artery of the heart of the object P. The apparatus obtains a left ventricular contrast-enhanced image by making the injector 30 inject a contrast medium from the catheter while continuously capturing X-ray fluoroscopy images with a region of interest including the heart while inserting the catheter into the left ventricle of the heart of the object P.

Assume that in this embodiment, the image storage unit 16 stores in advance many myocardial perfusion images and left ventricular contrast-enhanced images, together with the corresponding imaging times, which are obtained by performing the above imaging operation throughout a plurality of heartbeats without changing the irradiation range and direction of X-rays relative to the object P and without moving the top 5 or moving or rotating the C-arm 6.

### [Image Processing Unit]

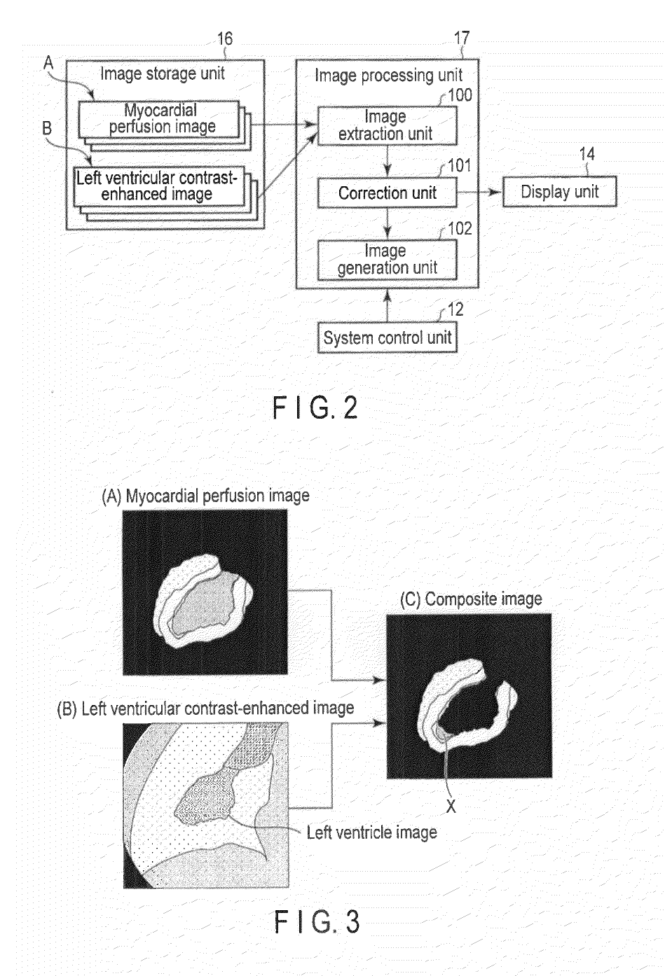

The function implemented by the image processing unit 17 will be described next. FIG. 2 is a block diagram for explaining the function of the image processing unit 17. FIG. 3 is a schematic view showing a procedure for image processing in this embodiment.

The image processing unit 17 in this embodiment implements functions as an image extraction unit 100, a correction unit 101, and an image generation unit 102 by executing computer programs stored in the memory or the like of the image processing unit 17 using a processor such as a CPU (Central Processing Unit).

The image extraction unit 100 extracts images to be used for combining operation and the like (to be described later) from a plurality of myocardial perfusion images (to be referred to as a myocardial perfusion image group A hereinafter) stored in the image storage unit 16 and a plurality of left ventricular contrast-enhanced images (to be referred to as a left ventricular contrast-enhanced image group B hereinafter) stored in the image storage unit 16.

Note that a myocardial perfusion image in this embodiment is obtained by the background subtraction processing of removing a background such as bones by subtracting a frame after the administration of a contrast medium from a frame before the administration of the contrast medium. As shown in FIG. 3(A), this image depicts the cardiac tissue contrast-enhanced by the contrast medium with higher luminance than other portions. In contrast, a left ventricular contrast-enhanced image in this embodiment is obtained without the above background subtraction processing. As shown in FIG. 3(B), this image depicts the left ventricle, and thoracic aorta contrast-enhanced by the contrast medium, with lower luminance than other portions.

The correction unit 101 performs various kinds of correction for the myocardial perfusion image and left ventricular contrast-enhanced image extracted by the image extraction unit and positions the respective images.

The image generation unit 102 generates a composite image like that shown in FIG. 3(C) by combining the myocardial perfusion image and left ventricular contrast-enhanced image corrected by the correction unit 101. Using this composite image allows to accurately comprehend the shape obtained by eliminating a portion corresponding to the left ventricle from the heart image depicted in a myocardial perfusion image, that is, the shape of the cardiac tissue. Of the shape of the cardiac tissue comprehended in this manner, for example, a lightly contrast-enhanced region like the portion indicated by symbol “X” in FIG. 3(C) is an ischemia region (including an infarction region).

The display unit 14 displays the composite image generated by the image generation unit 102 under the control of the system control unit 12.

### [Operation]

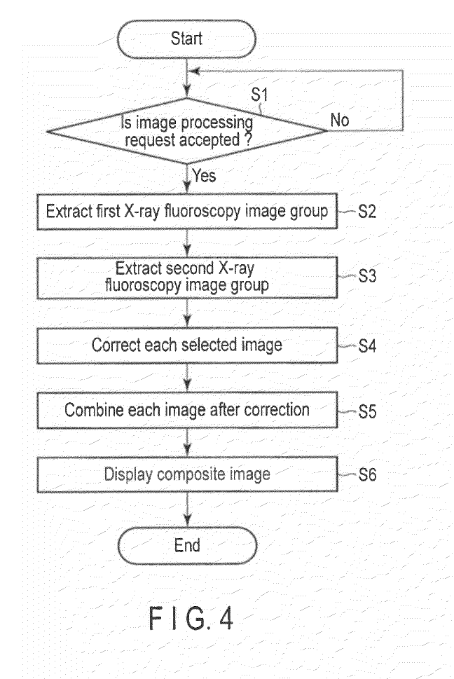

The concrete operation of the units 100 to 102 implemented by the image processing unit 17 and the system control unit 12 will be described next with reference to the flowchart of FIG. 4. Assume, as described above, that the image storage unit 16 has already stored the myocardial perfusion image group A and the left ventricular contrast-enhanced image group B.

As shown in the flowchart of FIG. 4, the system control unit 12 accepts an image processing request from the operator (step S1). The operator inputs an image processing request by, for example, operating the input unit 13. Upon accepting the image processing request (Yes in step S1), the system control unit 12 issues an instruction to start processing to the image processing unit 17.

When the system control unit 12 issues an instruction to start processing, the image extraction unit 100 extracts, from the myocardial perfusion image group A, an image group corresponding to one heartbeat in which the cardiac tissue of the object P is sufficiently contrast-enhanced (step S2). In addition, the image extraction unit 100 extracts, from the left ventricular contrast-enhanced image group B, an image group corresponding to one heartbeat in which the left ventricle of the object P is sufficiently contrast-enhanced (step S3). In other words, in steps S2 and S3, the image extraction unit 100 extracts myocardial perfusion images and left ventricular contrast-enhanced images corresponding to the same cardiac phase throughout one heartbeat. In the following description, the myocardial perfusion image group extracted in step S2 will be referred to as the first X-ray fluoroscopy image group, and the left ventricular contrast-enhanced image group extracted in step S3 will be referred to as the second X-ray fluoroscopy image group.

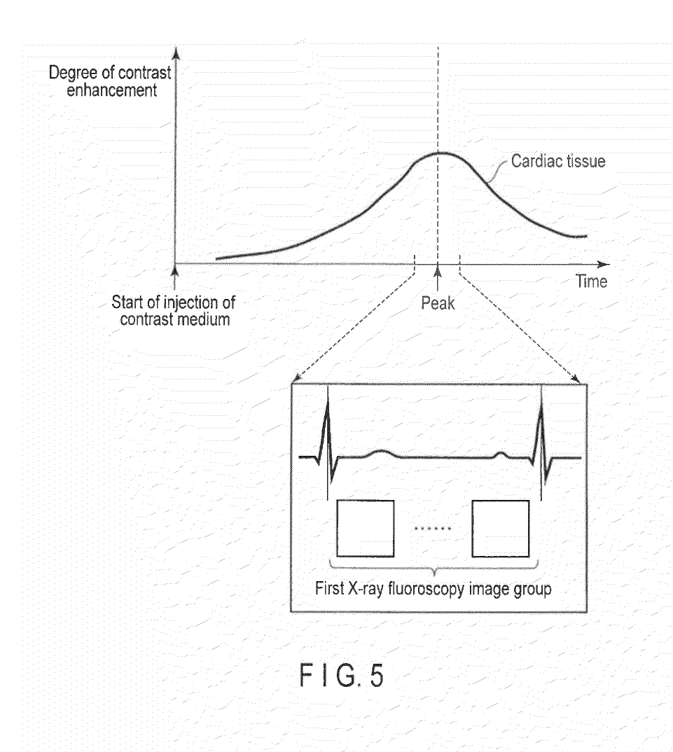

A technique of extracting the first X-ray fluoroscopy image group with the cardiac tissue being sufficiently contrast-enhanced in step S2 will be described with reference to FIG. 5. The contrast medium injected into a coronary artery at the time of capturing of a myocardial perfusion image flows into a blood vessel in the heart and then flows into the intercellular material of the cardiac tissue. At this time, the degree of contrast enhancement of the cardiac tissue in an X-ray fluoroscopy image after the contrast enhancement of the coronary artery gradually increases after the injection of the contrast medium and reaches the peak. Thereafter, the degree of contrast enhancement decreases.

In this embodiment, the image extraction unit 100 extracts a plurality of images in the range of one heartbeat centered on the above peak as the first X-ray fluoroscopy image group from the myocardial perfusion image group A. To implement this processing, the operator sets in advance, for example, a prediction time T1 by which the degree of contrast enhancement of the cardiac tissue reaches the peak from the time of injection of a contrast medium and a time width Wa corresponding to one heartbeat of the heart of the object P. The image extraction unit 100 then extracts, as the first X-ray fluoroscopy image group, a plurality of images, of the myocardial perfusion image group A, which are captured in the range of the time width Wa centered on the time point when the prediction time T1 has elapsed since the start time of injection of the contrast medium in coronary angiography which is stored in the image storage unit 16.

In a left ventricular contrast-enhanced image, the degree of contrast enhancement in the left ventricle gradually increases after the injection of the contrast medium, reaches the peak, and then decreases. Therefore, when extracting the second X-ray fluoroscopy image group in step S3, the operator also sets in advance a prediction time T2 by which the degree of contrast enhancement of the left ventricle reaches the peak from the time of injection of a contrast medium and a time width Wa corresponding to one heartbeat of the heart of the object P as in the case of the first X-ray fluoroscopy image group described with reference to FIG. 5. The image extraction unit 100 then extracts, as the second X-ray fluoroscopy image group, a plurality of images, of the left ventricular contrast-enhanced image group B, which are captured in the range of the time width Wa centered on the time point when the prediction time T2 has elapsed since the start time of injection of the contrast medium in left ventricle angiography which is stored in the image storage unit 16.

As a technique different from the above technique, the image extraction unit 100 may automatically specify a time by which the degree of contrast enhancement of the cardiac tissue reaches the peak and a time by which the degree of contrast enhancement of the left ventricle reaches the peak, based on a change in pixel value in each image included in the myocardial perfusion image group A and the left ventricular contrast-enhanced image group B. The image extraction unit 100 may automatically set the time width Wa based on the electrocardiographic waveform included in the phase information stored in the image storage unit 16.

In addition, the operator may manually extract the first and second X-ray fluoroscopy images from the myocardial perfusion image group A and the left ventricular contrast-enhanced image group B. In this case, for example, the display unit 14 may display a list of the myocardial perfusion image group A and left ventricular contrast-enhanced image group B. In this state, the image extraction unit 100 may accept the selection of a plurality of myocardial perfusion images and a plurality of left ventricular contrast-enhanced images by the operation with the input unit 13, extract the selected myocardial perfusion images as the first X-ray fluoroscopy image group, and extract the selected left ventricular contrast-enhanced images as the second X-ray fluoroscopy image group.

As in steps S2 and S3, after the first and second X-ray fluoroscopy image groups are extracted, the correction unit 101 performs various kinds of correction for each image included in the first and second X-ray fluoroscopy image groups (step S4). In this case, the correction includes, for example, the processing of adjusting the luminance value of each image included in the first X-ray fluoroscopy image group and the luminance value of each image included in the second X-ray fluoroscopy image group to values suitable for combining operation in step S5 (to be described later), and positioning (adjustment of positions, enlargement ratios, and image angles) of each image included in the first and second X-ray fluoroscopy image groups. The respective images may be positioned such that, for example, the shapes of regions with low X-ray transmittance, e.g., bones depicted in the respective images, match each other in the respective images. Alternatively, the operator may manually adjust the above luminance values or perform positioning.

After correction in step S4, the image generation unit 102 combines each image included in the first X-ray fluoroscopy image group after the correction with each image included in the second X-ray fluoroscopy image group after the correction to generate a composite image like that shown in FIG. 3(C) (step S5). More specifically, the image generation unit 102 generates a plurality of composite images corresponding to one heartbeat of the heart of the object P by combining the respective images included in the first and second X-ray fluoroscopy image groups, which have been captured in the same cardiac phases, by referring to phase information associated with each image. The image generation unit 102 performs this combining operation by, for example, adding pixel values of two images to be combined, which are located at the identical positions. Alternatively, the image generation unit 102 may calculate the averages of the pixel values of two images to be combined which are located at the identical positions.

After step S5, the system control unit 12 causes the display unit 14 to display the composite image generated by the image generation unit 102 (step S6), and terminates the series of processing. In step S6, the system control unit 12 may cause the display unit 14 to display, as still images, all or some of composite images corresponding one heartbeat which are generated by the image generation unit 102 or to display composite images corresponding to one heartbeat at a predetermined frame rate as a moving image. Alternatively, the input unit 13 may accept the selection of such a display form by the operator, and the apparatus may display a composite image in accordance with the selection.

As described above, the X-ray diagnostic apparatus 1 according to this embodiment generates a composite image like that shown in FIG. 3(C) by combining a myocardial perfusion image with a left ventricular contrast-enhanced image, and displays the image on the display unit 14. Referring to this composite image can clearly comprehend an ischemia region of the cardiac tissue of the object P.

In addition, since such a composite image is generated by using a myocardial perfusion image and a left ventricular contrast-enhanced image which correspond to the same cardiac phase of the object P, it is possible to comprehend an ischemia region with high accuracy.

Furthermore, this embodiment generates a composite image by using the myocardial perfusion image and the left ventricular contrast-enhanced image which are captured by the X-ray diagnostic apparatus. When the X-ray diagnostic apparatus captures these images, it is possible to administer a contrast medium into an object while capturing a fluoroscopic image of the object. This makes it easy to comprehend the spreading process of the contrast medium. In some ischemia region, contrast enhancement cannot be seen at an early stage after the administration of a contrast medium unlike other normal regions, but gradually appears afterward (delayed contrast enhancement). When using myocardial perfusion images and left ventricular contrast-enhanced images captured by the X-ray diagnostic apparatus as in this embodiment, it is easy to discriminate tissues in which such delayed contrast enhancement occurs as tissues with a risk of infarction and the like. Note that when, for example, obtaining myocardial perfusion images and left ventricular contrast-enhanced images by SPECT (Single Photon Emission Computed Tomography), since it takes time to perform imaging after the administration of a contrast medium into an object, even a tissue with the above risk may be contrast-enhanced at the time of imaging. This may make the doctor overlook such a tissue.

### Second Embodiment

The second embodiment will be described next.

This embodiment differs from the first embodiment in that it combines a trace image of the cardiac tissue depicted in each image included in the first X-ray fluoroscopy image group with a trace image of the left ventricle depicted in each image included in the second X-ray fluoroscopy image group instead of directly combining the respective images included in the first and second X-ray fluoroscopy image groups extracted by an image extraction unit 100.

This embodiment additionally has an arrangement for providing a doctor or the like with an image useful for a regenerative medical technique of injecting stem cells or cell growth factors into an ischemia region of a cardiac tissue by using the above composite trace image.

Assume that in this embodiment, in particular, the doctor inserts a catheter connected to an injector 30 into the body of an object P, makes an X-ray diagnostic apparatus 1 capture an X-ray fluoroscopy image of the heart of the object P and display it in real time, feeds the catheter into an ischemia region while seeing the image, and administers stem cells or the like from the distal end of the catheter when the distal end of the catheter reaches near the ischemia region.

Assume also that stem cells or the like to be administered from the catheter have been mixed with a contrast medium, and a region contrast-enhanced by the contrast medium will be depicted in the image displayed in real time at the time of administration of the stem cells or the like.

The same reference numerals denote the same constituent elements as those in the first embodiment, and a repetitive description will be made only when required.

### [Image Processing Unit]

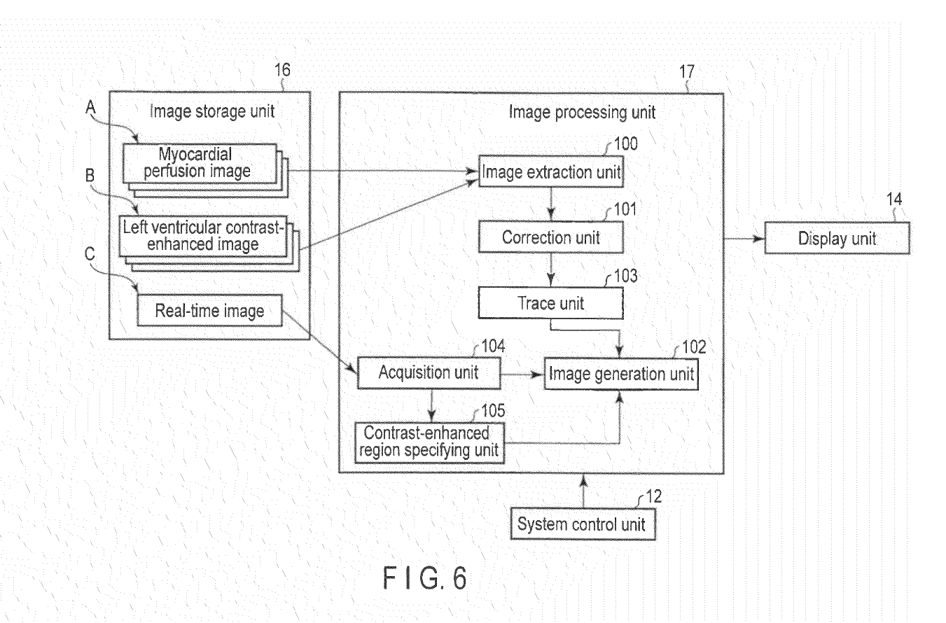

The overall arrangement of the X-ray diagnostic apparatus 1 according to this embodiment is the same as that shown in FIG. 1. Note, however, that an image processing unit 17 implements functions as a trace unit 103, an acquisition unit 104, and a contrast-enhanced region specifying unit 105 as shown in FIG. 6 in addition to the image extraction unit 100, the correction unit 101, and the image generation unit 102 shown in FIG. 2. The units 103 to 105 are also implemented by making a processor execute computer programs stored in a memory of the image processing unit 17.

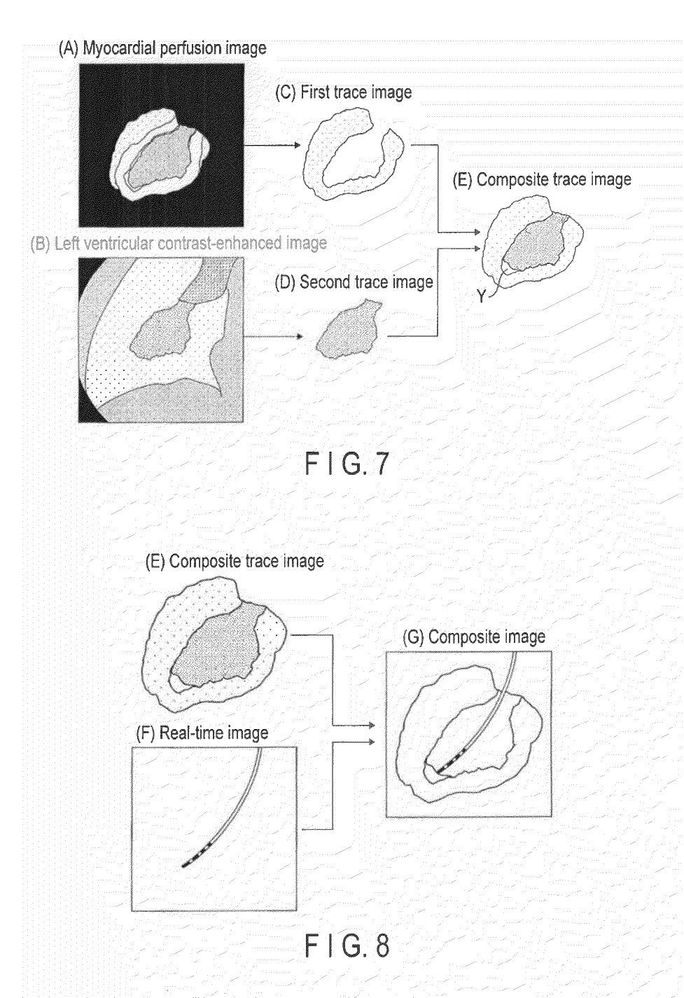

This embodiment performs image processing according to the procedures shown in FIGS. 7, 8, and 9.

The trace unit 103 traces the shape of the cardiac tissue depicted in a myocardial perfusion image extracted by the image extraction unit 100 and having undergone correction by the correction unit 101, and generates a trace image like that shown in FIG. 7(C). This trace image will be referred to as the first trace image hereinafter.

The trace unit 103 traces the shape of the left ventricle depicted in a left ventricular contrast-enhanced image extracted by the image extraction unit 100 and having undergone correction by the correction unit 101, and generates a trace image like that shown in FIG. 7(D). This trace image will be referred to as the second trace image hereinafter.

The acquisition unit 104 acquires real-time images C sequentially stored in an image storage unit 16. The real-time image C is a real-time X-ray fluoroscopy image which is continuously captured without changing the X-ray irradiation range and direction relative to the object P from the time of capturing the respective images included in a myocardial perfusion image group A and left ventricular contrast-enhanced image group B and without moving a top 5 or moving or rotating a C-arm 6. The real-time image C is captured when administering stem cells or the like into the object P. When the catheter is inserted near the heart of the object P, the catheter is depicted in the real-time image C, as shown in FIG. 8(F).

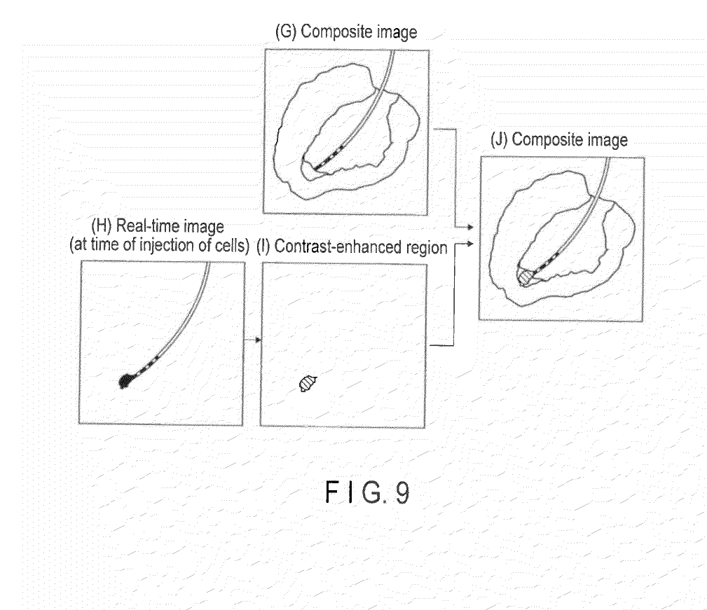

The contrast-enhanced region specifying unit 105 specifies a region (to be referred to as a contrast-enhanced region hereinafter) contrast-enhanced by a contrast medium mixed in stem cells or the like from the real-time images C sequentially acquired by the acquisition unit 104, as shown in FIGS. 9(H) and 9(I).

Note that the image generation unit 102 in this embodiment generates a composite trace image by combining the first trace image with the second trace image, as shown in FIG. 7(E).

While the acquisition unit 104 is acquiring the real-time images C, the image generation unit 102 sequentially generates images by placing the composite trace images of the first and second trace images on the real-time images C acquired by the acquisition unit 104, as shown in FIGS. 8(E) and 8(G).

When a contrast medium is injected into the object P, the image generation unit 102 generates an image by placing the composite trace image on the real-time image C, and sequentially generates images with the regions specified by the contrast-enhanced region specifying unit 105 being segmented, as shown in FIGS. 9(G) and 9(J).

Note that FIG. 8(G) and FIGS. 9(G) and 9(J) exemplify a case in which only line segments representing the contours of the first and second trace images included in a composite image are arranged on the real-time image C. However, the inside portions surrounded by the line segments may be colored in predetermined colors. Alternatively, the first and second trace images included in a composite trace image may be colored in predetermined colors, respectively, without using the above line segments, and may be arranged on the real-time image C. In addition, when coloring the first and second trace images, it is possible to make the background (real-time image C) transmissive at a predetermined transmittance.

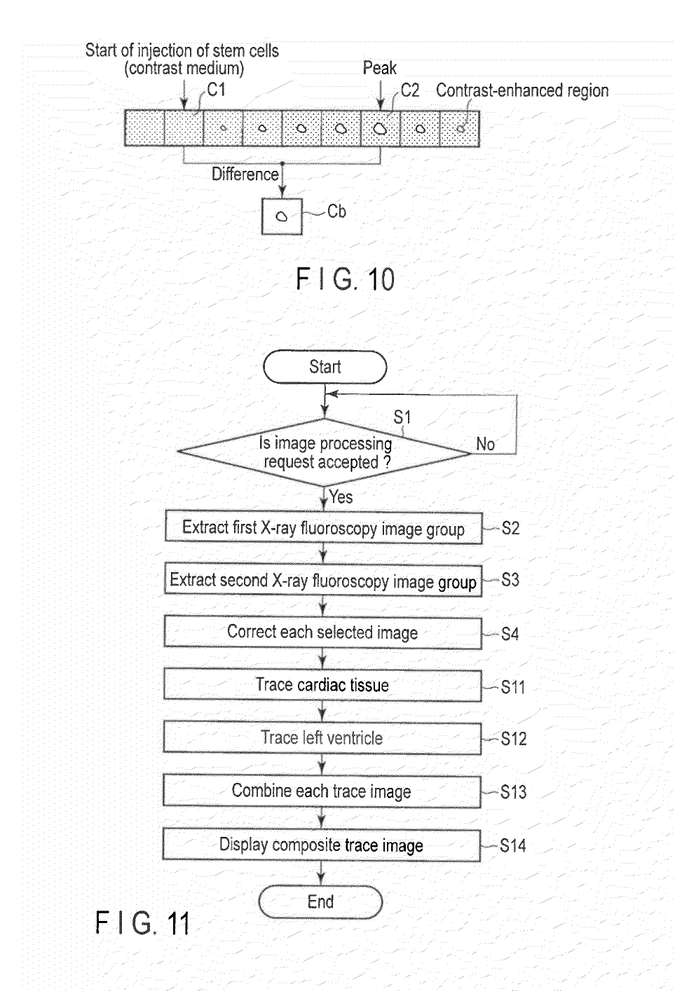

A procedure by which the contrast-enhanced region specifying unit 105 specifies the above contrast-enhanced region will be described below with reference to FIG. 10. At the time of administration of stem cells or the like, the above contrast-enhanced region depicted in the real-time image C spreads with the lapse of time, reaches the peak, and gradually disappears, as shown in FIG. 10.

The contrast-enhanced region specifying unit 105 according to this embodiment generates, first of all, a difference image Cd between a real-time image C1 captured at the start time of administration of stem cells or the like and a real-time image C2 captured at the time corresponding to the above peak. This subtraction erases the catheter, bones, and the like depicted in the real-time image C2. The contrast-enhanced region specifying unit 105 then regards the high-luminance region depicted in the difference image Cd as the above contrast-enhanced region.

Note that it is possible to use, as the real-time image C1, the real-time image C captured at the time when, for example, an instruction to start injecting stem cells or the like is issued to the injector 30. It is also possible to use, as the real-time image C2, the real-time image C captured at the time when a prediction time T3 set in advance, which is the time interval between the start time of administration of, for example, stem cells or the like and the time when the spread of a contrast-enhanced region reaches the peak, has elapsed since the time when an instruction to start injecting stem cells or the like is issued to the injector 30. Alternatively, the contrast-enhanced region specifying unit 105 may automatically set the real-time images C1 and C2 by image processing for the real-time image C.

The concrete operations of the units 100 to 105 implemented by the image processing unit 17 and system control unit 12 will be described next. Assume that as in the first embodiment, the image storage unit 16 stores in advance the myocardial perfusion image group A and the left ventricular contrast-enhanced image group B.

[Operation before Administration of Stem Cells or The Like]

This embodiment executes the processing shown in the flowchart of FIG. 11 instead of the processing shown in the flowchart of FIG. 4.

Processing in steps S1 to S4 shown in the flowchart of FIG. 11 is the same as that described in the first embodiment. That is, first of all, the system control unit 12 accepts an image processing request from the operator (step S1). When the system control unit 12 accepts an image processing request (Yes in step S1), the image extraction unit 100 extracts the first X-ray fluoroscopy image from the myocardial perfusion image group A (step S2), and further extracts the second X-ray fluoroscopy image group from the left ventricular contrast-enhanced image group B (step S3). The correction unit 101 then performs various kinds of correction for each image included in the first and second X-ray fluoroscopy image groups (step S4).

After step S4, the trace unit 103 performs processing in this embodiment. That is, the trace unit 103 generates the first trace image of each image included in the first X-ray fluoroscopy image group after the correction by tracing the shape of the cardiac tissue depicted in each image (step S11). In this processing, the trace unit 103 may generate the first trace image like that shown in FIG. 7(C) by, for example, extracting a high-luminance region depicted in a myocardial perfusion image of a processing target and tracing the shape of the extracted region.

The trace unit 103 generates the second trace image of each image included in the second X-ray fluoroscopy image group after the correction by tracing the shape of the left ventricle depicted in each image (step S12). In this processing, the trace unit 103 may generate the second trace image like that shown in FIG. 7(D) by, for example, extracting a low-luminance region depicted near the center of a left ventricular contrast-enhanced image of the processing target and tracing the shape obtained by removing portions corresponding to the thoracic aorta and the aortic valve from the extracted low-luminance region.

Note that in steps S11 and S12, the operator may generate the first trace image by manually tracing the cardiac tissue depicted in each image included in the first X-ray fluoroscopy image group, and may generate the second trace image by tracing the left ventricle depicted in each image included in the second X-ray fluoroscopy image group.

Upon completion of tracing of all the images included in the first and second X-ray fluoroscopy image groups in steps S11 and S12, the image generation unit 102 combines each first trace image and each second trace image, and generates an image on which the composite trace image is placed (step S13). More specifically, the image generation unit 102 combines each first trace image and each second trace image generated in steps S11 and S12 which match in cardiac phase to generate an image on which the composite trace image is placed throughout one heartbeat, as shown in FIG. 7(E). Note that it is possible to specify the cardiac phase of each trace image by referring to phase information associated with an X-ray fluoroscopy image based on which each trace image is generated.

After step S13, the system control unit 12 causes the display unit 14 to display the image generated by the images generation unit 102 (step S14), and terminates the series of processing. In step S14, the system control unit 12 may cause the display unit 14 to display, as still images, all or some of composite images corresponding to one heartbeat which are generated by the image generation unit 102, or may cause the display unit 14 to display, as a moving image, the images corresponding to one heartbeat. Alternatively, the input unit 13 may accept the selection of such a display form by the operator, and the apparatus may display a composite image in accordance with the selection.

In an image displayed in this manner, the region surrounded by the first and second trace images (a region Y in FIG. 7) can be estimated as a region of the cardiac tissue in which no blood is supplied, i.e., a region of the cardiac tissue in which ischemia (including infarction) has occurred.

### [Operation at Time of Administration of Stem Cells]

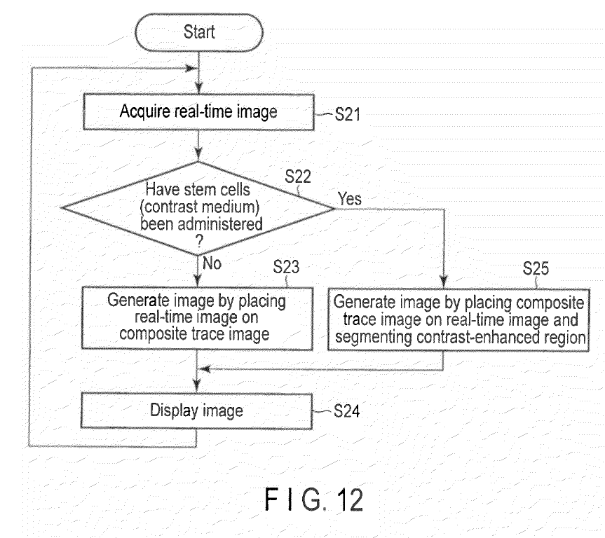

When the doctor inserts a catheter into the object P to administer stem cells to a cardiac tissue of the object P and makes the X-ray diagnostic apparatus 1 start capturing the real-time image C described above, each unit of the image processing unit 17 and the system control unit 12 execute the processing shown in the flowchart of FIG. 12. Note that in parallel with this processing, the contrast-enhanced region specifying unit 105 executes the processing for specifying the contrast-enhanced region described above to specify the contrast-enhanced region depicted in the real-time image C.

In the flowchart shown in FIG. 12, first of all, the acquisition unit 104 acquires the latest real-time image C stored in the image storage unit 16 (step S21). When the doctor inserts the catheter into the object P, the catheter is depicted in the real-time image C, as shown in FIG. 8(F).

Subsequently, the image generation unit 102 determines whether there is a region to which stem cells or the like has completely been administered through the catheter (step S22). The image generation unit 102 performs this determination by determining whether there is a contrast-enhanced region specified by the contrast-enhanced region specifying unit 105 after the start of the processing shown in the flowchart.

At the stage where no stem cells have been administered through a catheter, there is no contrast-enhanced region specified by the contrast-enhanced region specifying unit 105 (No in step S22). In this case, the image generation unit 102 generates an image by placing the composite image generated in step S13 on the real-time image C acquired in step S21 (step S23), as shown in FIG. 8(G). In this processing, the apparatus may select an arbitrary one of the composite trace images corresponding to one heartbeat which are generated in step S13, and may combine the selected composite trace image with the real-time image C. For example, the doctor or the like designates a composite image to be selected in advance before the processing shown in the flowchart. Alternatively, the image generation unit 102 may automatically select one of composite trace images corresponding to one heartbeat which corresponds to a specific cardiac phase.

After step S23, the system control unit 12 causes a display unit 14 to display the composite image generated by the image generation unit 102 (step S24). The process then returns to step S21 to execute processing in steps S21 to 24 for the real-time image C captured next and stored in the image storage unit 16.

Repeating the processing in steps S21 to S24 in this manner will display an image like that shown in FIG. 8(G) on the display unit 14 in real time. The doctor may move the catheter while referring to this picture, and position the distal end of the catheter to an ischemia region of the cardiac tissue, i.e., the region surrounded by the first and second trace images.

When the distal end of the catheter reaches the ischemia region thereafter, the doctor administers stem cells or the like into the body of the object P through the catheter. At this time, as described above, the contrast-enhanced region specifying unit 105 specifies the region contrast-enhanced by the contrast medium mixed in the stem cells or the like.

After a contrast-enhanced region is specified, the apparatus determines in step S22 that there is a region to which the stem cells or the like have completely been administered (Yes in step S22). In this case, the image generation unit 102 generates an image by placing the composite trace image generated in step S13 on the real-time image C acquired in step S21 and segmenting the contrast-enhanced region specified by the contrast-enhanced region specifying unit 105 (step S25), as shown in FIG. 9(J). Note that a composite trace image to be used in this case may be selected by the same method as that in step S23.

The image generation unit 102 segments a contrast-enhanced region by, for example, making it have a color or pattern different from that of other regions included in the real-time image C. Alternatively, the image generation unit 102 may segment a contrast-enhanced region by, for example, placing a line segment indicating the shape of the region on the real-time image C.

After step S25, the system control unit 12 causes the display unit 14 to display the composite image generated by the image generation unit 102 (step S24).

Once stem cells or the like are administered, the apparatus repeatedly executes these processes in the order of steps S21, S22, S25, and S24. As a result, an image like that shown in FIG. 9(J) is displayed on the display unit 14 in real time. Referring to this picture allows the doctor to comprehend the ischemia region to which no stem cells or the like have been administered.

Note that when stem cells or the like are injected a plurality of number of times after the start of the processing shown in the flowchart, the contrast-enhanced region specifying unit 105 specifies the contrast-enhanced region corresponding to each injecting operation. In this case, in step S25, the image generation unit 102 generates an image with a contrast-enhanced region corresponding to each injecting operation being segmented.

In this case, the composite trace image to be combined with the real-time image C in steps S23 and S25 may differ each time the processing in steps S23 and S25 is executed. For example, a composite trace image to be combined with the real-time image C is selected such that a cardiac phase of the heart of the object P at the time of capturing the real-time image C to be combined matches a cardiac phase corresponding to a myocardial perfusion image and left ventricular contrast-enhanced image based on which a composite trace image is generated. This makes the composite trace image in the image displayed on the display unit 14 in real time pulsate in accordance with the actual cardiac phases of the object P. In addition, in this case, the apparatus may decrease the frame rate of images displayed on the display unit 14 to sequentially display only composite images corresponding to specific cardiac phases.

As described above, the X-ray diagnostic apparatus 1 according to this embodiment combines the first trace image obtained by tracing the cardiac tissue with the second trace image obtained by tracing the left ventricle and displays the composite trace image on the display unit 14. Referring to the composite trace image displayed in this manner allows to accurately and easily comprehend an ischemia region of the cardiac tissue.

In addition, the X-ray diagnostic apparatus 1 according to this embodiment generates an image by placing the above composite trace image on the real-time image C, and displays the generated image on the display unit 14 in real time. Seeing the image displayed in this manner makes it possible to clearly comprehend the position of the distal end of the catheter inserted into the body of the object P and the position that the distal end should reach, i.e., an ischemia region of the cardiac tissue.

The X-ray diagnostic apparatus 1 according to this embodiment generates an image by segmenting the region to which stem cells or the like have completely been administered on the real-time image C, and displays the generated image on the display unit 14 in real time. Seeing the image displayed in this manner allows to easily comprehend the region to which stem cells or the like have completely been administered.

### Third Embodiment

The third embodiment will be described next.

This embodiment differs from the first and second embodiments in that it specifies an ischemia region of a cardiac tissue based on the composite trace image described in the second embodiment, segments a specified ischemia region on a real-time image C, and erases a portion to which stem cells or the like have completely been administered from the region segmented in this manner.

The same reference numerals denote the same constituent elements as those in the first and second embodiments, and a repetitive description will be made only when required.

### [Image Processing Unit]

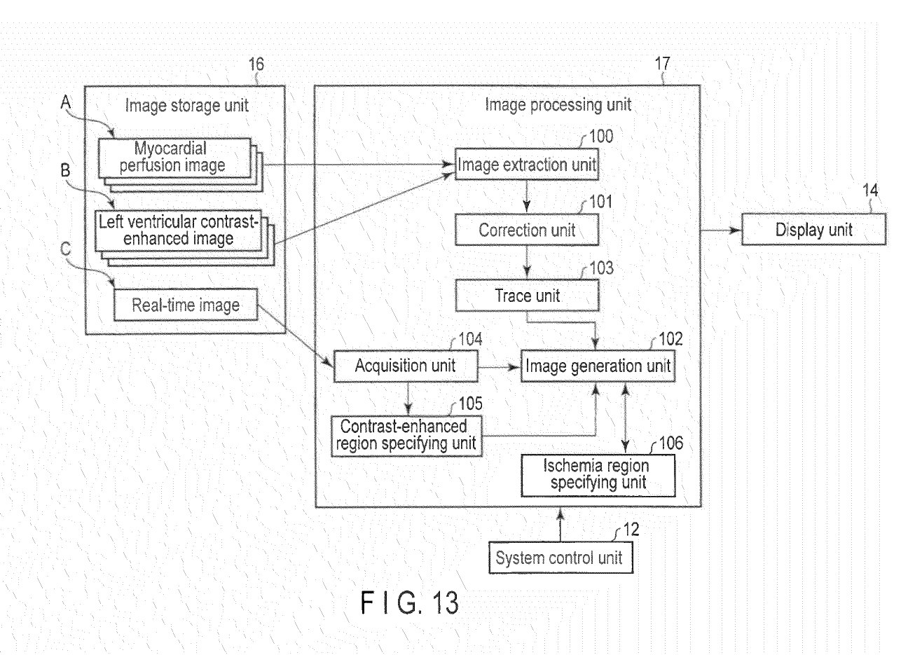

The overall arrangement of an X-ray diagnostic apparatus 1 according to this embodiment is the same as that shown in FIG. 1. Note, however, that an image processing unit 17 implements a function as an ischemia region specifying unit 106 as shown in FIG. 13, in addition to the image extraction unit 100, the correction unit 101, the image generation unit 102, the trace unit 103, the acquisition unit 104, and the contrast-enhanced region specifying unit 105 which are shown in FIG. 6. The ischemia region specifying unit 106 is also implemented by making a processor execute a computer program stored in a memory or the like of the image processing unit 17.

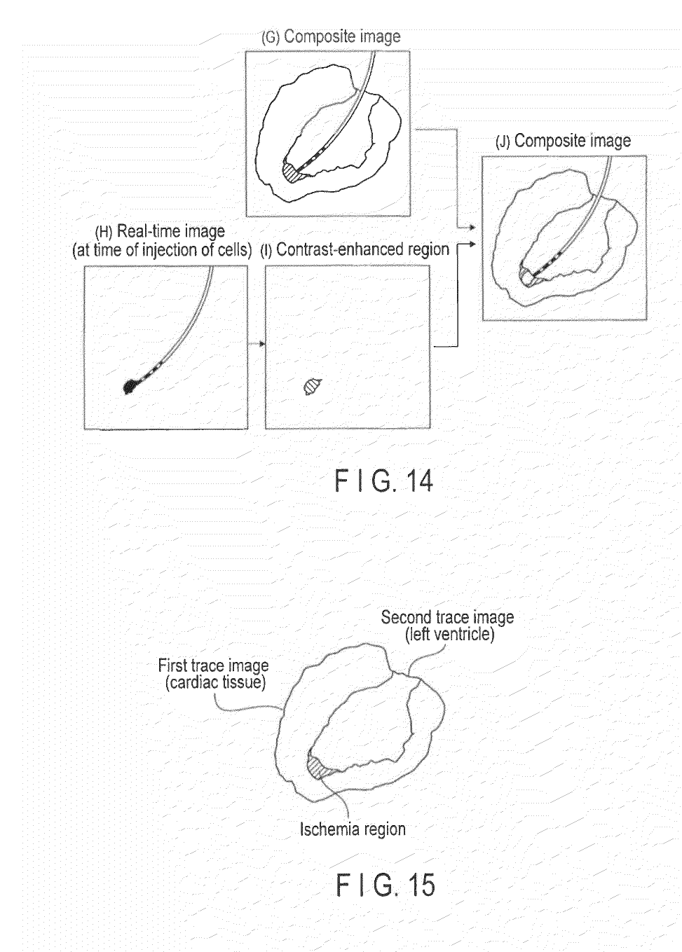

This embodiment performs image processing according to the procedure shown in FIG. 14.

The ischemia region specifying unit 106 specifies an ischemia region of a cardiac tissue of an object P based on a myocardial perfusion image group A and a left ventricular contrast-enhanced image group B which are stored in an image storage unit 16. More specifically, the ischemia region specifying unit 106 regards, as an ischemia region of the cardiac tissue, the region (the hatched portion in FIG. 15) surrounded by the first trace image representing the shape of the cardiac tissue and the second trace image representing the shape of the left ventricle in the composite trace image generated by the image generation unit 102 according to the procedure described in the second embodiment as shown in FIG. 15.

The image generation unit 102 in this embodiment generates an image on which the composite trace image of the first and second trace images is placed and the ischemia region specified by the 10 is segmented, as shown in FIG. 15.

While the acquisition unit 104 acquires real-time images C, the image generation unit 102 sequentially generates images by placing the composite trace images on the real-time images C acquired by the acquisition unit 104 and segmenting the ischemia region specified by the ischemia region specifying unit 106, as shown in FIG. 14(G).

When a contrast medium is injected into the object P, the image generation unit 102 sequentially generates an image by placing the composite trace image on the real-time image C acquired by the acquisition unit 104 and segmenting a portion, of the ischemia region specified by the ischemia region specifying unit 106, which does not overlap the contrast-enhanced region specified by the contrast-enhanced region specifying unit 105, as shown in FIG. 14(J).

[Operation before Administration of Stem Cells or the Like]

Like the second embodiment, this embodiment executes the processing shown in the flowchart of FIG. 11.

Note, however, that when the image generation unit 102 generates composite trace images throughout one heartbeat by using the first and second trace images in step S13, the ischemia region specifying unit 106 specifies an ischemia region of each of the composite trace images by the above technique. The image generation unit 102 further generates an image on which a composite trace image is placed and the ischemia region specified based on the composite trace image is segmented for each of the composite trace images throughout one heartbeat. Each ischemia region may be segmented by, for example, making it have a color or pattern different from that of other regions included in the real-time image C.

After step S13, a system control unit 12 causes a display unit 14 to display the images generated by the image generation unit 102 (step S14), and terminates the series of processing. In step S14, the system control unit 12 may cause the display unit 14 to display, as still images, all or some of images corresponding one heartbeat which are generated by the image generation unit 102 or to display the images corresponding to one heartbeat at a predetermined frame rate as a moving image. Alternatively, the input unit 13 may accept the selection of such a display form by the operator, and the apparatus may display an image in accordance with the selection. Referring to the image displayed in this manner allows to accurately and easily comprehend an ischemia region of the cardiac tissue.

### [Operation at Time of Administration of Stem Cells or The Like]

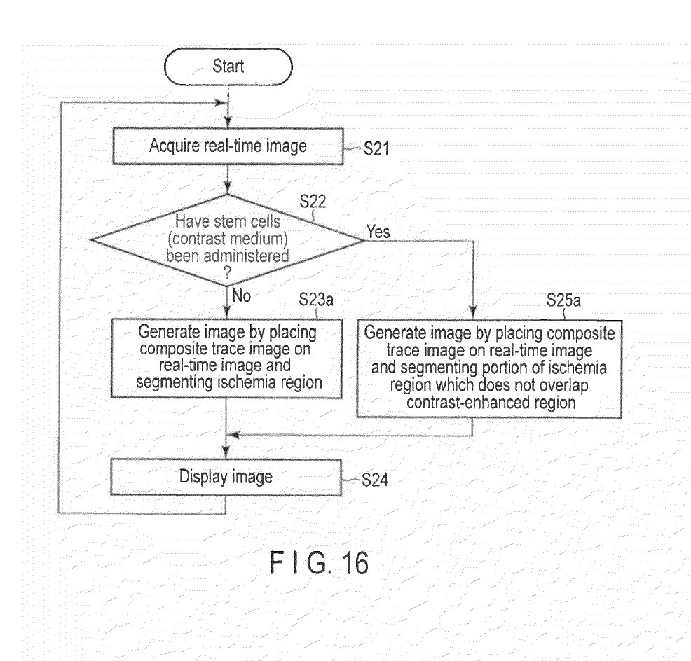

In this embodiment, when the apparatus starts capturing the real-time image C, the respective units of the image processing unit 17 and the system control unit 12 execute the processing shown in the flowchart of FIG. 16. Note that in parallel with this processing, the contrast-enhanced region specifying unit 105 executes the processing for specifying the contrast-enhanced region described in the second embodiment to specify the contrast-enhanced region depicted in the real-time image C.

In the flowchart shown in FIG. 16, first of all, as described in the second embodiment, the acquisition unit 104 acquires the latest real-time image C stored in the image storage unit 16 (step S21). The image generation unit 102 then determines whether there is a region to which stem cells or the like have completely been administered through the catheter (step S22).

At the stage where no stem cells have been administered through a catheter, there is no contrast-enhanced region specified by the contrast-enhanced region specifying unit 105 (No in step S22). In this case, as shown in FIG. 14(G), the image generation unit 102 generates an image by placing the predetermined composite image generated in step S13 on the real-time image C acquired in step S21 and segmenting the ischemia region specified by the ischemia region specifying unit 106 (step S23a). In this processing, the apparatus may select an arbitrary one of the composite trace images corresponding to one heartbeat which are generated in step S13, and may segment the ischemia region specified based on the composite trace image while placing the selected combine composite trace image on the real-time image C. For example, the doctor or the like designates a composite image to be selected in advance before the processing shown in the flowchart. Alternatively, the image generation unit 102 may automatically select one of composite trace images corresponding to one heartbeat which corresponds to a specific cardiac phase. In addition, each ischemia region is segmented by, for example, making it have a color or pattern different from that of other regions included in the real-time image C. Alternatively, the apparatus may segment a contrast-enhanced region by, for example, placing a line segment indicating the shape of the region on the real-time image C.

After step S23a, the system control unit 12 causes the display unit 14 to display the composite image generated by the image generation unit 102 (step S24). The process then returns to step S21 to execute processing in steps S21, S22, S23a, and S24 for the real-time image C captured next and stored in the image storage unit 16.

Repeating the processing in steps S21, S22, S23a, and S24 in this manner will display an image like that shown in FIG. 14(G) on the display unit 14 in real time.

When stem cells or the like are administered into the body of the object P through the catheter, the contrast-enhanced region specifying unit 105 specifies the region contrast-enhanced by the contrast medium mixed in the stem cells or the like, as described above.

After the contrast-enhanced region is specified, the apparatus determines in step S22 that there is a region into which the stem cells have completely been injected (Yes in step S22). In this case, as shown in FIG. 14(J), the image generation unit 102 generates an image by placing the composite trace image generated in step S13 on the real-time image C acquired in immediately preceding step S21 and segmenting a portion, of the ischemia region specified by the ischemia region specifying unit 106, which does not overlap the contrast-enhanced region specified by the contrast-enhanced region specifying unit 105 (step S25a). Note that a composite trace image to be used in this case may be selected by the same method as that in step S23a. The apparatus segments a non-overlapping portion between an ischemia region and a contrast-enhanced region by, for example, making it have a color or pattern different from that of other regions included in the real-time image C. Alternatively, the apparatus may segment the non-overlapping portion by, for example, placing a line segment indicating the shape of the region on the real-time image C.

As is obvious from FIG. 14(J), after step S25a, the apparatus erases the contrast-enhanced region, i.e., the region to which stem cells or the like have already been administered, from the region segmented before the administration of the stem cells or the like.

After step S25a, the system control unit 12 causes the display unit 14 to display the composite image generated by the image generation unit 102 (step S24).

Once stem cells or the like are administered, the apparatus repeatedly executes these processes in the order of steps S21, S22, S25a, and S24. As a result, an image like that shown in FIG. 14(J) is displayed on the display unit 14 in real time.

Note that when stem cells or the like are injected a plurality of times after the start of the processing shown in the flowchart, the contrast-enhanced region specifying unit 105 specifies the contrast-enhanced region corresponding to each injecting operation. In this case, in step S25a, the image generation unit 102 generates an image by segmenting a portion, of the ischemia region, which does not overlap either contrast-enhanced region in each injecting operation.

In this case, the composite trace image to be combined with the real-time image C in steps S23a and S25a may differ each time the processing in steps S23a and S25a is executed. For example, a composite trace image to be combined with the real-time image C is selected such that a cardiac phase of the heart of the object P at the time of capturing the real-time image C to be combined matches a cardiac phase corresponding to a myocardial perfusion image and left ventricular contrast-enhanced image based on which a composite trace image is generated. This makes the composite trace image and ischemia region in the image displayed on the display unit 14 in real time pulsate in accordance with the actual cardiac phases of the object P. In addition, in this case, the apparatus may decrease the frame rate of images displayed on the display unit 14 to sequentially display only a composite image corresponding to a specific cardiac phase.

As described above, the X-ray diagnostic apparatus 1 according to this embodiment generates an image by segmenting an ischemia region of the cardiac tissue of the object P on an image on which the first and second trace images are arranged or the real-time image C, and displays the generated image on the display unit 14. Referring to this image allows to easily comprehend the above ischemia region.

In addition, when stem cells or the like are injected into the body of the object P, the X-ray diagnostic apparatus 1 according to this embodiment generates an image by segmenting a portion, of the ischemia region, which does not overlap the region to which stem cells or the like have been administered, and displays the generated image on the display unit 14. Referring to this image allows to easily comprehend the portion to which no stem cells or the like have been administered.

### Forth Embodiment

In the first to third embodiments, it is assumed that the image storage unit 16 stores in advance the myocardial perfusion image group A and left ventricular contrast-enhanced image group B captured by the X-ray diagnostic apparatus 1.

When, however, specifying an ischemia/infarction region, it is not always necessary to use the myocardial perfusion image group A and left ventricular contrast-enhanced image group B captured by the X-ray diagnostic apparatus 1.

In the arrangements of the first to third embodiments, it is possible to use the images captured by modalities, other than an X-ray diagnostic apparatus, e.g., an X-ray CT (Computed Tomography) apparatus, SPECT apparatus, MRI (Magnetic Resonance Imaging) apparatus, ultrasonic diagnostic apparatus, and PET (Positron Emission Tomography) apparatus, instead of the myocardial perfusion image group A and the left ventricular contrast-enhanced image group B.

In the arrangements disclosed in the first to third embodiments, when using the images captured by modalities other than an X-ray diagnostic apparatus, a myocardial perfusion image group A′ (first images) and a left ventricular contrast-enhanced image group B′ (second images) are stored in the image storage unit 16 in advance instead of the myocardial perfusion image group A and left ventricular contrast-enhanced image group B captured by the X-ray diagnostic apparatus 1.

In step S2, an image extraction unit 100 extracts, from the myocardial perfusion image group A′, an image group corresponding to one heartbeat in which the cardiac tissue of an object P is sufficiently contrast-enhanced. In addition, in step S3, the image extraction unit 100 extracts, from the left ventricular contrast-enhanced image group B′, an image group corresponding to one heartbeat in which the left ventricle of the object P is sufficiently contrast-enhanced. In this embodiment, the myocardial perfusion image group extracted in step S2 will be referred to as the first image group, and the left ventricular contrast-enhanced image group extracted in step S3 will be referred to as the second image group.

A procedure for processing using the first and second image groups is the same as that for the processing described in the first to third embodiments.

In the first embodiment, a correction unit 101 performs various kinds of correction for each image included in the first and second image groups (step S4). An image generation unit 102 generates a composite image like that shown in FIG. 3(C) by combining each image of the first image group after the correction with each image of the second image group after the correction (step S5). Thereafter, a system control unit 12 causes a display unit 14 to display the composite image generated by the image generation unit 102 (step S6).

In the second embodiment, the correction unit 101 performs various kinds of correction for each image included in the first and second image groups (step S4), and the trace unit 103 generates the first trace image by tracing the shape of the cardiac tissue depicted in each image included in the first image group after the correction (step S11). The trace unit 103 also generates the second trace image by tracing the shape of the left ventricle depicted in each image included in the second image group after the correction (step S12). When the trace unit 103 completes tracing for all the images included in the first and second image groups in steps S11 and S12, the image generation unit 102 combines each first trace image with each second trace image, and generates an image in which the composite trace image is placed (step S13). Thereafter, the system control unit 12 causes the display unit 14 to display the image generated by the image generation unit 102 (step S14).

At the time of administration of stem cells or the like, the acquisition unit 104 acquires the latest real-time image C stored in the image storage unit 16 (step S21). The image generation unit 102 then determines whether there is a region to which stem cells or the like have completely been administered through the catheter (step S22). If there is no region to which stem cells or the like have completely been administered (No in step S22), the image generation unit 102 generates an image by placing the composite trace image generated in step S13 on the real-time image C acquired in step S21 (step S23). The system control unit 12 then causes the display unit 14 to display the composite image generated by the image generation unit 102 (step S24). If there is a region to which stem cells or the like have been administered (Yes in step S22), the image generation unit 102 generates an image by placing the composite trace image generated in step S13 on the real-time image C acquired in step S21 and segmenting the contrast-enhanced region specified by the contrast-enhanced region specifying unit 105 (step S25). The system control unit 12 then causes the display unit 14 to display the composite image generated by the image generation unit 102 (step S24).

In the third embodiment, upon determining, at the time of administration of stem cells or the like that there is no region to which stem cells or the like have completely been administered through the catheter (No in step S22), the image generation unit 102 generates an image by placing the predetermined composite image generated in step S13 on the real-time image C acquired in step S21 and segmenting the ischemia region specified by the ischemia region specifying unit 106 (step S23a). The system control unit 12 causes a display unit 14 to display the composite image generated by the image generation unit 102 in this manner (step S24). Upon determining that there is a region to which stem cells or the like have completely been administered (Yes in step S22), the image generation unit 102 generates an image by placing the composite trace image generated in step S13 on the real-time image C acquired in immediately preceding step S21 and segmenting a portion, of the ischemia region specified by the ischemia region specifying unit 106, which does not overlap the contrast-enhanced region specified by the contrast-enhanced region specifying unit 105 (step S25a). The system control unit 12 then causes the display unit 14 to display the composite image generated by the image generation unit 102 in this manner (step S24).

Note that each modification concerning the second and third embodiments can use the following arrangement.

That is, the trace unit 103 generates the third trace image by tracing an ischemia region in each cardiac phase based on the first and second image groups. The third trace image may be generated by, for example, tracing the region surrounded by the first and second trace images which is regarded as an ischemia region. Alternatively, the third trace image may be generated by tracing the ischemia region specified by the image obtained by combining images included in the first and second image groups which correspond to the same cardiac phase.

In the second embodiment, in step S23, the image generation unit 102 generates an image by placing the third trace image on the real-time image C acquired in step S21. In step S25, the image generation unit 102 generates an image by placing the third trace image on the real-time image C acquired in step S21 and segmenting the contrast-enhanced region specified by the contrast-enhanced region specifying unit 105.

In the third embodiment, in step S23a, the image generation unit 102 generates an image by placing the third trace image on the real-time image C acquired in step S21 and segmenting the ischemia region specified by the ischemia region specifying unit 106. In step S25a, the image generation unit 102 generates an image by placing the third trace image on the real-time image C acquired in immediately preceding step S21 and segmenting a portion, of the ischemia region specified by the ischemia region specifying unit 106, which does not overlap the contrast-enhanced region specified by the contrast-enhanced region specifying unit 105.

As described above, even by using the myocardial perfusion image group A′ and left ventricular contrast-enhanced image group B′ captured by a modality other than an X-ray diagnostic apparatus, it is possible to obtain the same effects as those in the first to third embodiments.

### Modification

In the arrangement disclosed in each embodiment described above, the respective constituent elements can be modified and embodied at the execution stage.

The following are concrete modifications.

(1) Each embodiment described above has exemplified the case in which the X-ray diagnostic apparatus 1 incorporates constituent elements concerning the image processing unit 17, the image storage unit 16, and the like. However, image processing described in each embodiment may be implemented by an image processing apparatus other than the X-ray diagnostic apparatus 1.

(2) A myocardial perfusion image used in image processing in each embodiment described above may be an image obtained by removing a portion other than the cardiac tissue by background subtraction. In this case, the apparatus may generate difference images based on, for example, X-ray fluoroscopy images captured immediately before the injection of a contrast medium into a coronary artery and X-ray fluoroscopy images which are sequentially captured thereafter and depict a contrast-enhanced region, and generate a myocardial perfusion image group based on these difference images. In addition, each image included in a myocardial perfusion image group may be a combination of an image obtained from the left coronary artery upon injecting a contrast medium and an image obtained from the right coronary artery upon injecting the contrast medium.

(3) Each embodiment described above uses left ventricular contrast-enhanced images in image processing. In addition to left ventricular contrast-enhanced images, however, the apparatus may use a combination of images obtained by contrast enhancement imaging of the left atrium, right ventricle, and right atrium in image processing. In this case, for example, in the first embodiment, the apparatus may generate composite images using these four kinds of cardiac lumen contrast-enhanced images and myocardial perfusion images. In the second embodiment, the apparatus may generate trace images of left ventricle, left atrium, right ventricle, and right atrium and combine the images based on the four types of cardiac lumen contrast-enhanced images with trace images of the cardiac tissue. In addition, in the third embodiment, it is possible to regard, as an ischemia region, the region surrounded by the trace images of left ventricle, left atrium, right ventricle, and right atrium and the trace images of the cardiac tissue.

(4) In each embodiment described above, the image extraction unit 100 extracts images corresponding to one heartbeat from a myocardial perfusion image group and a left ventricular contrast-enhanced image group. However, the image extraction unit 100 may extract first and second X-ray fluoroscopy images (or first and second images) one by one from the respective image groups. In this case, the apparatus may generate a composite image or trace image by using first and second X-ray fluoroscopy images (or first and second images) one by one.

(5) Each embodiment described above has exemplified the case in which image processing is performed by using myocardial perfusion images and left ventricular contrast-enhanced images captured from a single direction. If, however, the X-ray diagnostic apparatus has an arrangement capable of acquiring images from multiple directions, e.g., a biplane system, the apparatus may perform the image processing described in each embodiment by using myocardial perfusion images and left ventricular contrast-enhanced images captured from the respective directions.

(6) The second to fourth embodiments are based on the assumption that a catheter is inserted into the body of the object P and fed to an ischemia region region, and stem cells or the like are administered from the distal end of the catheter. However, the arrangements disclosed in these embodiments are also useful when stem cells or the like are administered to an ischemia region by other methods. Other methods include a method of perforating a small hole in the body surface of the object P, inserting a tube into the hole, and feeding stem cells or the like from the surface of the heart of the object P through the tube instead of through a blood vessel.

(7) In each embodiment described above, the processor of the image processing unit 17 implements the functions of the units 100 to 106 and the like by executing the computer programs stored in the memory. However, each embodiment is not limited to this and may download the above computer programs from a predetermined network into the X-ray diagnostic apparatus 1 or may store similar functions in a recording medium and installing them in the X-ray diagnostic apparatus 1. As a recording medium, it is possible to use a CD-ROM, USB memory, or the like. In addition, a recording medium in any form can be used as long as a device incorporated in or connected to the X-ray diagnostic apparatus 1 can read. Furthermore, the functions obtained by installing or downloading programs in advance in this manner may be implemented in cooperation with the OS (Operating System) in the X-ray diagnostic apparatus 1 and the like.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing apparatus comprising:
a storage unit configured to store a first X-ray fluoroscopy image with a cardiac tissue of an object being contrast-enhanced by a contrast medium and a second X-ray fluoroscopy image with a cardiac lumen of the object being contrast-enhanced by the contrast medium;
an image generation unit configured to generate an image by combining the first X-ray fluoroscopy image and the second X-ray fluoroscopy image which are stored in the storage unit; and
a display control unit configured to cause a display unit to display the image generated by the image generation unit.

2. The image processing apparatus of claim 1, wherein the storage unit stores the plurality of first X-ray fluoroscopy images and the plurality of second X-ray fluoroscopy images which are captured in a chronological order,
the apparatus further comprises an image extraction unit configured to extract the first X-ray fluoroscopy image and the second X-ray fluoroscopy image corresponding to the same cardiac phase from the images stored in the storage unit, and
the image generation unit generates an image by combining the first X-ray fluoroscopy image and the second X-ray fluoroscopy image which are extracted by the image extraction unit.

3. An image processing apparatus comprising:
a storage unit configured to store a first X-ray fluoroscopy image with a cardiac tissue of an object being contrast-enhanced by a contrast medium and a second X-ray fluoroscopy image with a cardiac lumen of the object being contrast-enhanced by the contrast medium;
a trace unit configured to generate a first trace image by tracing a shape of a cardiac tissue depicted in the first X-ray fluoroscopy image stored in the storage unit and generate a second trace image by tracing a shape of the cardiac lumen depicted in the second X-ray fluoroscopy image stored in the storage unit;
an image generation unit configured to generate an image by combining the first trace image and the second trace image which are generated by the trace unit; and
a display control unit configured to cause a predetermined display unit to display the image generated by the image generation unit.

4. The image processing apparatus of claim 3, wherein the storage unit stores the plurality of first X-ray fluoroscopy images and the plurality of second X-ray fluoroscopy images which are captured in a chronological order,
the apparatus further comprises an image extraction unit configured to extract the first X-ray fluoroscopy image and the second X-ray fluoroscopy image corresponding to the same cardiac phase from the images stored in the storage unit, and
the trace unit generates the first trace image by tracing a shape of a cardiac tissue depicted in the first X-ray fluoroscopy image extracted by the image extraction unit and generates the second trace image by tracing a shape of the cardiac lumen depicted in the second X-ray fluoroscopy image extracted by the image extraction unit.

5. The image processing apparatus of claim 4, further comprising an acquisition unit configured to sequentially acquire real-time X-ray fluoroscopy images of the object which are captured by an X-ray diagnostic apparatus configured to capture an X-ray fluoroscopy image,
wherein the image generation unit combines the first trace image and the second trace image generated by the trace unit, and sequentially generates images by placing trace images after combining on X-ray fluoroscopy images sequentially acquired by the acquisition unit.

6. The image processing apparatus of claim 5, further comprising a contrast-enhanced region specifying unit configured to specify a region contrast-enhanced by a contrast medium and depicted in an X-ray fluoroscopy image acquired by the acquisition unit,
wherein the image generation unit combines the first trace image and the second trace image which are generated by the trace unit, and sequentially generates images by placing trace images after combining on X-ray fluoroscopy images sequentially acquired by the acquisition unit and segmenting regions specified by the contrast-enhanced region specifying unit.

7. An image processing apparatus comprising:
a storage unit configured to store a first X-ray fluoroscopy image with a cardiac tissue of an object being contrast-enhanced by a contrast medium and a second X-ray fluoroscopy image with a cardiac lumen of the object being contrast-enhanced by the contrast medium;
an ischemia region specifying unit configured to specify an ischemia region of the cardiac tissue based on the first X-ray fluoroscopy image and the second X-ray fluoroscopy image which are stored in the storage unit;
an image generation unit configured to generate an image by segmenting an ischemia region specified by the ischemia region specifying unit on a predetermined image concerning the heart of the object; and
a display control unit configured to cause a predetermined display unit to display the image generated by the image generation unit.

8. The image processing apparatus of claim 7, further comprising a trace unit configured to generate a first trace image by tracing a shape of a cardiac tissue depicted in the first X-ray fluoroscopy image stored in the storage unit and generate a second trace image by tracing a shape of the cardiac lumen depicted in the second X-ray fluoroscopy image stored in the storage unit,
wherein when the first trace image and the second trace image which are generated by the trace unit are combined, the ischemia region specifying unit specifies, as the ischemia region, a region surrounded by the respective trace images.

9. The image processing apparatus of claim 8, wherein the storage unit stores the plurality of first X-ray fluoroscopy images and the plurality of second X-ray fluoroscopy images which are captured in a chronological order,
the apparatus further comprises an image extraction unit configured to extract the first X-ray fluoroscopy image and the second X-ray fluoroscopy image corresponding to the same cardiac phase from the images stored in the storage unit, and
the trace unit generates the first trace image by tracing a shape of a cardiac tissue depicted in the first X-ray fluoroscopy image extracted by the image extraction unit and generates the second trace image by tracing a shape of the cardiac lumen depicted in the second X-ray fluoroscopy image extracted by the image extraction unit.

10. The image processing apparatus of claim 8, wherein the image generation unit generates an image by combining and placing the first trace image and the second trace image which are generated by the trace unit and segmenting an ischemia region specified by the ischemia region specifying unit.

11. The image processing apparatus of claim 7, further comprising an acquisition unit configured to sequentially acquire real-time X-ray fluoroscopy images of the object which are captured by an X-ray diagnostic apparatus configured to capture an X-ray fluoroscopy image,
wherein the image generation unit sequentially generates images by segmenting ischemia regions specified by the ischemia region specifying unit on X-ray fluoroscopy images sequentially acquired by the acquisition unit.

12. The image processing apparatus of claim 11, further comprising a contrast-enhanced region specifying unit configured to specify a region contrast-enhanced by a contrast medium and depicted in an X-ray fluoroscopy image acquired by the acquisition unit,
wherein the image generation unit sequentially generates images by segmenting portions, of ischemia regions specified by the ischemia region specifying unit, which do not overlap regions specified by the contrast-enhanced region specifying unit on X-ray fluoroscopy images sequentially acquired by the acquisition unit.

13. An image processing apparatus comprising:
an acquisition unit configured to sequentially acquire real-time X-ray fluoroscopy images of an object which are captured by an X-ray diagnostic apparatus;
an image generation unit configured to sequentially generate images by placing, on X-ray fluoroscopy images sequentially acquired by the acquisition unit, composite images of first trace images, each obtained by tracing a shape of a cardiac tissue depicted in first image with a cardiac tissue of the object being contrast-enhanced by a contrast medium, and second trace images, each obtained by tracing a shape of the cardiac lumen depicted in a second image with a cardiac lumen of the object being contrast-enhanced by a contrast medium, or third trace images each obtained by tracing an ischemia region specified from a composite image of the first image and the second image; and
a display control unit configured to cause a predetermined display unit to display the image generated by the image generation unit.

14. An X-ray diagnostic apparatus comprising:
an X-ray imagine unit configured to capture a first X-ray fluoroscopy image with a cardiac tissue of an object being contrast-enhanced by a contrast medium and a second X-ray fluoroscopy image with a cardiac lumen of the object being contrast-enhanced by the contrast medium;
a storage unit configured to store the first X-ray fluoroscopy image and the second X-ray fluoroscopy image which are captured by the X-ray imaging unit;
an image generation unit configured to generate an image by combining the first X-ray fluoroscopy image and the second X-ray fluoroscopy image which are stored in the storage unit; and
a display control unit configured to cause a display unit to display the image generated by the image generation unit.

15. The X-ray diagnostic apparatus of claim 14, wherein the storage unit stores the plurality of first X-ray fluoroscopy images and the plurality of second X-ray fluoroscopy images which are captured in a chronological order,
the apparatus further comprises an image extraction unit configured to extract the first X-ray fluoroscopy image and the second X-ray fluoroscopy image corresponding to the same cardiac phase from the images stored in the storage unit, and
the image generation unit generates an image by combining the first X-ray fluoroscopy image and the second X-ray fluoroscopy image which are extracted by the image extraction unit.
